(19) United States
(12) Patent Application Publication
(10) Pub. No.: US 2014/0154304 A1
(43) Pub. Date: Jun. 5, 2014

(54) COMBINATION THERAPY WITH VOLASERTIB

(71) Applicant: Tillmann TAUBE, Biberach an der Riss (DE)

(72) Inventor: Tillmann TAUBE, Biberach an der Riss (DE)

(73) Assignee: BOEHRINGER INGELHEIM INTERNATIONAL GMBH, Ingelheim am Rhein (DE)

(21) Appl. No.: 14/088,948

(22) Filed: Nov. 25, 2013

(30) Foreign Application Priority Data
Nov. 30, 2012 (EP) 12195162.8

(51) Int. Cl. A61K 31/519; A61K 31/704; A61K 31/7068; A61K 38/19; A61K 31/7076

(52) U.S. Cl. CPC A61K 31/519; A61K 38/193; A61K 31/7076; A61K 31/7068; A61K 31/704
USPC 424/450; 424/85.1

(57) ABSTRACT

Disclosed is the use of Volasertib or a salt or a hydrate thereof for treating patients suffering from acute myeloid leukemia (AML) comprising administering a high dose of Volasertib in combination with fludarabine, cytarabine and Granulocyte colony-stimulating factor (GCSF) or in combination with fludarabine, cytarabine, GCSF and a daunorubicin citrate liposome injection.

The present invention relates to the use of Volasertib or a salt thereof or the hydrate thereof for treating patients suffering from acute myeloid leukemia (AML) comprising a high dose of Volasertib administered in combination with fludarabine, cytarabine and Granulocyte colony-stimulating factor (GCSF) or in combination with fludarabine, cytarabine, GCSF and a daunorubicin citrate liposome injection.

### BACKGROUND OF THE INVENTION

Acute myeloid leukemia (AML), also known as acute myelogenous leukemia, is a cancer of the myeloid line of blood cells, characterized by the rapid growth of abnormal white blood cells that accumulate in the bone marrow and interfere with the production of normal blood cells. As an acute leukemia, AML progresses rapidly and is typically fatal within weeks or months if left untreated. AML is the most prevalent form of adult leukemia, particularly among the elderly and is slightly more common in men than women. There is an estimated prevalence of 30,000 cases of AML in the US and 47,000 in the EU.

The incidence of AML increases with age with a median age at diagnosis of 67 years. The global incidence CAGR for AML out to 2013 is 1.4%. An aging population, along with an increased incidence of treatment-related AML in cancer survivors, currently accounting for 10-20% of all AML cases, is expected to drive the incidence of AML. In addition, there is some geographic variation in the incidence of AML. In adults, the highest rates are seen in North America, Europe, and Oceania, while adult AML is rarer in Asia and Latin America.

AML accounts for approximately 1.2% of all cancer deaths. The 5 year survival rates for AML are low, driven by therapy failure and patients relapsing. Among patients <65 the 5 year survival rate is 34.4%, among patients >65 it is only 5%.

According to the French-American-British (FAB) classification system AML is divided into subtypes (M0 to M8), based on the type of cell from which the leukemia developed and its degree of maturity. The WHO classification incorporates of genetic abnormalities into diagnostic algorithms for the diagnosis of AML. This classification is done by examining the appearance of the malignant cells under light microscopy and by using cytogenetics and molecular genetics to characterize any underlying chromosomal abnormalities or genetic changes. The subtypes impact on prognoses, responses to therapy and treatment decisions.

The WHO subtypes are as follows:

Acute Myeloid Leukemia and Related Neoplasms
Acute myeloid leukemia with recurrent genetic abnormalities
   AML with t(8;21)(q22;q22); RUNX1-RUNX1T1
   AML with inv(16)(p13.1q22) or t(16;16)(p13.1;q22); CBFB-MYH11
   APL with t(15;17)(q22;q12); PML-RARA
   AML with t(9;11)(p22;q23); MLLT3-MLL
   AML with t(6;9)(p23;q34); DEK-NUP214
   AML with inv(3)(q21 q26.2) or t(3;3)(q21;q26.2); RPN1-EVI1
   AML (megakaryoblastic) with t(1;22)(p13;q13); RBM15-MKL1
   Provisional entity: AML with mutated NPM1
   Provisional entity: AML with mutated CEBPA
Acute myeloid leukemia with myelodysplasia-related changes
Therapy-related myeloid neoplasms
Acute myeloid leukemia, not otherwise specified
   AML with minimal differentiation
   AML without maturation
   AML with maturation
   Acute myelomonocytic leukemia
   Acute monoblastic/monocytic leukemia
   Acute erythroid leukemia
      Pure erythroid leukemia
      Erythroleukemia, erythroid/myeloid
   Acute megakaryoblastic leukemia
   Acute basophilic leukemia
   Acute panmyelosis with myelofibrosis
Myeloid sarcoma
Myeloid proliferations related to Down syndrome
   Transient abnormal myelopoiesis
   Myeloid leukemia associated with Down syndrome
Blastic plasmacytoid dendritic cell neoplasm

The efficacy of chemotherapeutic agents can be improved by improving the dosage schedule and/or using combination therapies with other compounds. Even if the concept of combining several therapeutic agents or improved dosage schedules already has been suggested, there is still a need for new and efficient therapeutic concepts for the treatment of cancer diseases, which show advantages over standard therapies.

Volasertib is a highly potent and selective inhibitor of the serine-threonine Polo like kinase 1 (Plk1), a key regulator of cell-cycle progression. Volasertib is a second-generation dihydropteridinone derivative with distinct pharmacokinetic (PK) properties. The problem underlying this invention was:
a) to develop combinations of Volasertib with fludarabine, cytarabine and GCSF and improved dosage schedules thereof for the treatment of AML with maximal activity and limited toxicity.
b) to develop combinations of Volasertib with fludarabine, cytarabine, GCSF and a daunorubicin citrate liposome injection and improved dosage schedules thereof for the treatment of AML with maximal activity and limited toxicity.

Volasertib (I) is known as the compound N-[trans-4-[4-(cyclopropylmethyl)-1-piperazinyl]cyclohexyl]-4-[[(7R)-7-ethyl-5,6,7,8-tetrahydro-5-methyl-8-(1-methylethyl)-6-oxo-2-pteridinyl]amino]-3-methoxy-benzamide,

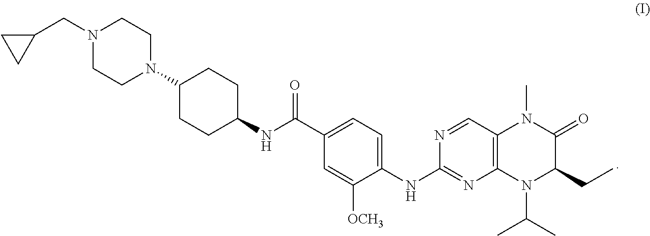

This compound is disclosed in WO 04/076454. Furthermore, trihydrochloride salt forms and hydrates thereof are known from WO 07/090844. They possess properties which make those forms especially suitable for pharmaceutical use. The above mentioned patent applications further disclose the use of this compound or its monoethanesulfonate salt for the preparation of pharmaceutical compositions intended especially for the treatment of diseases characterized by excessive or abnormal cell proliferation.

Fludarabine (Fludara®) is a purine analog, and can be given both orally and intravenously. Fludarabine inhibits DNA synthesis by interfering with ribonucleotide reductase and DNA polymerase. It is active against both dividing and resting cells. Being phosphorylated, fludarabine is ionized at physiologic pH and is effectually trapped in blood. This provides some level of specificity for blood cells, both cancerous and healthy.

Cytarabine is inter alia known by the brand names Cytosar-U, Tarabine PFS, DepoCyte and AraC. Cytarabine is mainly used in the treatment of acute myeloid leukaemia, acute lymphocytic leukaemia (ALL) and in lymphomas.

Granulocyte colony-stimulating factor (GCSF) is a colony-stimulating factor hormone. GCSF is also known as colony-stimulating factor 3 (CSF 3). It is a glycoprotein, growth factor and cytokine produced by a number of different tissues to stimulate the bone marrow to produce granulocytes and stem cells. GCSF then stimulates the bone marrow to release them into the blood. GCSF also stimulates the survival, proliferation, differentiation, and function of neutrophil precursors and mature neutrophils. GCSF regulates them using Janus kinase (JAK)/signal transducer and activator of transcription (STAT) and Ras/mitogen-activated protein kinase (MAPK) and phosphatidylinositol 3-kinase (Pl3K)/protein kinase B (Akt) signal transduction pathway. It was first marketed by Amgen with the brand name Neupogen. Several generic versions are now also available. The recombinant human GCSF is called filgrastim and available under the name Neupogen. PEG-filgrastim (Neulasta) are two commercially-available forms of recombinant human GCSF. The PEG polyethylene glycol (PEG) form has a much longer half-life, reducing the necessity of daily injections. Another form of recombinant human GCSF called lenograstim is synthesised in CHO cells. As this is a mammalian cell expression system, lenograstim is indistinguishable from the 174-amino acid natural human GCSF.

DaunoXome® (daunorubicin citrate liposome injection) is a prescription drug indicated as a first line cytotoxic therapy for advanced HIV-associated Kaposi's sarcoma. It belongs to a class of drugs known as anthracyclines and works by slowing or stopping the growth of cancer cells.

### SUMMARY OF THE INVENTION

The present invention relates to a new combination for the treatment of a patient suffering from AML wherein Volasertib is administered in combination with
a) fludarabine, cytarabine and GCSF, or
b) with fludarabine, cytarabine, GCSF and a daunorubicin citrate liposome injection.

Accordingly, a first object of the present invention refers to a method of treating AML or for treatment of a patient suffering from AML by administration to the patient suffering from AML
a) an effective amount (50-500 μg/m² body surface area, preferably 200 μg/m² by i.v. infusion) of GCSF at day 0, 1, 2, 3, 4 and 5 during a 6 day treatment cycle (days 0 to 5),
b) an effective amount (10-100 mg/m² body surface area, preferably 30 mg/m²) of fludarabine at day 1, 2, 3, 4 and 5 during said 6 day treatment cycle,
c) an effective amount (500-4000 mg/m² body surface area, preferably 2000 mg/m²) of cytarabine at day 1, 2, 3, 4 and 5 during said 6 day treatment cycle, and
d) an effective amount (10 to 350 mg/m² body surface area, preferably 10, 50, 100, 150, 200, 250, 300, or 350 mg/m²) of Volasertib or a pharmaceutically acceptable salt thereof or a hydrate thereof at at least one day and up to 5 days during said 6 day treatment cycle.
At day 15 (counted from day 0 of above mentioned 6 day treatment cycle) GCSF administration is restarted with the same dose as indicated under a) above for so many days until neutrophil recovery.
After neutrophils have recovered the therapy can be restarted at day 0.

Another object of the present invention refers to a method of treating AML comprising administration to a patient suffering from AML
a) an effective amount (50-500 μg/m² body surface area, preferably 200 μg/m² by i.v. infusion) of GCSF at day 0, 1, 2, 3, 4 and 5 during a 6 day treatment cycle (days 0 to 5),
b) an effective amount (10-100 mg/m² body surface area, preferably 30 mg/m²) of fludarabine at day 1, 2, 3, 4 and 5 during said 6 day treatment cycle,
c) an effective amount (500-4000 mg/m² body surface area, preferably 2000 mg/m²) of cytarabine at day 1, 2, 3, 4 and 5 during said 6 day treatment cycle,
d) an effective amount (500-4000 mg/m² body surface area, preferably 2000 mg/m²) of daunorubicin citrate liposome at day 1, 3 and 5 during said 6 day treatment cycle, and
e) an effective amount (10 to 350 mg/m² body surface area, preferably 10, 50, 100, 150, 200, 250, 300, or 350 mg/m²) of Volasertib or a pharmaceutically acceptable salt thereof or a hydrate thereof at at least one day and up to 5 days during said 6 day treatment cycle.
At day 15 (counted from day 0 of above mentioned 6 day treatment cycle) GCSF administration is restarted with the same dose as indicated under a) above for so many days until neutrophil recovery.
After neutrophils have recovered the therapy can be restarted at day 0.

Both bombination treatments can be supported by intrathecal medication (e.g. on day 0).

Another object of the present invention is a method of treating AML in patients suffering from AML wherein Volasertib or a pharmaceutically acceptable salt thereof or a hydrate thereof is administered at day 1, 2, 3, 4 and 5 during said 6 day treatment cycle.

Another object of the present invention is a method of treating AML in patients suffering from AML wherein Volasertib or a pharmaceutically acceptable salt thereof or a hydrate thereof is administered at day 1, 2, 3 and 4 during said 6 day treatment cycle.

Another object of the present invention is a method of treating AML in patients suffering from AML wherein Volasertib or a pharmaceutically acceptable salt thereof or a hydrate thereof is administered at day 1, 2 and 3 during said 6 day treatment cycle.

Another object of the present invention is a method of treating AML in patients suffering from AML wherein Volasertib or a pharmaceutically acceptable salt thereof or a hydrate thereof is administered at day 1 and 2 during said 6 day treatment cycle.

Another object of the present invention is a method of treating AML in patients suffering from wherein Volasertib or a pharmaceutically acceptable salt thereof or a hydrate thereof is administered at day 1 during said 6 day treatment cycle.

Another object of the invention refers to Volasertib or a pharmaceutically acceptable salt thereof or a hydrate thereof for use in a method to treat AML in a patient suffering from AML characterized in that Volasertib or a pharmaceutically acceptable salt thereof or a hydrate thereof is administered according to one of the combination treatment described above.

Another object of the invention refers to fludarabine for the use in treating AML in patients suffering from AML characterized in that Volasertib or a pharmaceutically acceptable salt thereof or a hydrate thereof is administered according to one of the combination treatment described above.

Another object of the invention refers to cytarabine for the use in treating AML in patients suffering from AML characterized in that Volasertib or a pharmaceutically acceptable salt thereof or a hydrate thereof is administered according to one of the combination treatment described above.

Another object of the invention refers to GCSF for the use in treating AML in patients suffering from AML characterized in that Volasertib or a pharmaceutically acceptable salt thereof or a hydrate thereof is administered according to one of the combination treatment described above.

Another object of the invention refers to daunorubicin citrate liposome for the use in treating AML in patients suffering from AML characterized in that Volasertib or a pharmaceutically acceptable salt thereof or a hydrate thereof is administered according to one of the combination treatment described above.

Another object of the invention refers to the use of Volasertib or a pharmaceutically acceptable salt thereof or a hydrate thereof for the manufacture of a medicament for treating AML in patients suffering from AML wherein the medicament is prepared for administration according to one of the combination treatment described above.

Another object of the invention refers to the use of fludarabine for the manufacture of a medicament for treating AML in patients suffering from AML wherein the medicament is prepared for administration according to one of the combination treatment described above.

Another object of the invention refers to the use of cytarabine for the manufacture of a medicament for treating AML in patients suffering from AML wherein the medicament is prepared for administration according to one of the combination treatment described above.

Another object of the invention refers to the use of GCSF for the manufacture of a medicament for treating AML in patients suffering from AML wherein the medicament is prepared for administration according to one of the combination treatment described above.

Another object of the invention refers to the use of daunorubicin citrate liposome for the manufacture of a medicament for treating AML in patients suffering from AML wherein the medicament is prepared for administration according to one of the combination treatment described above.

Another object of the invention is a pharmaceutical composition comprising an effective amount of Volasertib and an effective amount of fludarabine, cytarabine and GCSF together with an instruction for administration of the active ingredients to a patient suffering from AML.

Another object of the invention is a pharmaceutical composition comprising an effective amount of Volasertib and an effective amount of fludarabine, cytarabine, daunorubicin citrate liposome and GCSF together with an instruction for administration of the active ingredients to a patient suffering from AML.

Another object of the present invention is the compound Volasertib for use in coadministration with fludarabine, cytarabine, daunorubicin citrate liposome and GCSF to a patient suffering from AML, characterized in that Volasertib is administered according to the above mentioned combination treatment.

Another object of the present invention is the compound Volasertib for use in coadministration with fludarabine, cytarabine and GCSF to a patient suffering from AML, characterized in that Volasertib is administered according to the above mentioned combination treatment.

Another object of the present invention is the use of Volasertib for preparation of a pharmaceutical composition comprising an effective amount of Volasertib fludarabine, cytarabine, daunorubicin citrate liposome and GCSF together with an instruction for administration of the active ingredients to a patient suffering from AML, wherein Volasertib is administered according to the above mentioned combination treatment.

Another object of the present invention is the use of Volasertib for preparation of a pharmaceutical composition comprising an effective amount of Volasertib fludarabine, cytarabine and GCSF together with an instruction for administration of the active ingredients to a patient suffering from AML, wherein Volasertib is administered according to the above mentioned combination treatment.

### DETAILED DESCRIPTION OF THE INVENTION

For example, the administration of Volasertib at at least one day and up to 5 days during a 6 day treatment cycle means that Volasertib can be administered once or up to 5 times during said period, wherein only one dosage is administered per day. For example it might be administered at day 1 only, or it can be administered at day 1, 3 and 5. It might also be administered at days 1 to 5 or at day 1 and 5 only.

The above described treatment can be repeated as long as patients are eligible for repeated cycles, i.e. until progression of disease and as long as neither patient nor investigator requests treatment discontinuation.

The instruction for coadministration may be in any form suitable for pharmaceuticals, e.g. in form of a leaflet added to the dosage form within secondary packaging or an imprint on the primary or secondary packaging.

The skilled in the art is aware that it may optionally be necessary to deviate from the dosage amounts specified for Volasertib, fludarabine, cytarabine, daunorubicin citrate liposome and GCSF, depending on the body weight or method of administration, the individual response to the medication, the nature of the formulation used and the time or interval over which it is administered. Thus, in some cases, it may be sufficient to use less than the minimum quantity specified above, while in other cases the upper limit specified will have to be exceeded. When large amounts are administered it may be advisable to spread them over the day in a number of single doses. For example, in intensive treatment schedules up to 4000 mg/m² body surface area of cytarabine can be administered.

Dosage Forms and Formulation Aspects

Regarding any aspects of the invention for Volasertib pharmaceutically acceptable salts or hydrates thereof may be used, preferably trihydrochloride salt forms and hydrates thereof as disclosed in WO 07/090844. Dosages or amounts of the actives provided in the context of this invention refer in any case to the free base equivalent, that is Volasertib in the free base form.

The term “therapeutically effective amount” shall mean that amount of a drug or pharmaceutical agent that will elicit the biological or medical response of a tissue system, animal or human that is being sought by a researcher or clinician, resulting in a beneficial effect for at least a statistically significant fraction of patients, such as a improvement of symptoms, a cure, a reduction in disease load, reduction in tumor mass or leukaemia cell numbers, extension of life, or improvement in quality of life.

Day 0 of a 6 day treatment cycle is defined as that day at which the first dose of GCSF is administered.

The above indicated dosage regimens are especially useful to treat human patients suffering from AML being of an age of 18 years or younger.

The term “relapsed AML” is defined as reappearance of leukaemic blasts in the blood or >5% blasts in the bone marrow after CR (complete remission) not attributable to any other cause. For patients presenting with relapsed AML, >5% blasts on baseline bone marrow assessment is required.

The term “refractory AML” is defined as a failure to achieve a CR or CRi (complete remission with incomplete blood recovery) after previous therapy. Any number of prior anti-leukemia schedules is allowed.

The term “complete remission” is defined as morphologically leukaemia free state (i.e. bone marrow with <5% blasts by morphologic criteria and no Auer rods, no evidence of extramedullary leukaemia) and absolute neutrophil count ≧1,000/μL and platelets >100,000/μL.

The term “complete remission with incomplete blood recovery” is defined as morphologically leukaemia free state (i.e. bone marrow with <5% blasts by morphologic criteria and no Auer rods, no evidence of extramedullary leukaemia) and neutrophil count <1,000/μL or platelets <100,000/μL in the blood.

AML patients who are considered ineligible for intensive treatment constitute an accepted subgroup although no validated algorithm has been established to determine a patient's eligibility for intensive treatment. As reflected in current practice guidelines (NCCN Clinical practice Guidelines in Oncology™, Acute Myeloid Leukemia V.2.2021), the patient's age and duration of previous remission are important variables to assess a patient's eligibility for intensive treatment. However, many other factors will contribute to the medical assessment (e.g. AML cytogenetics, performance status, prior stem cell transplantation, concomitant diagnoses). Thus, an assessment of ineligibility for intensive treatment is required to ensure a defined and homogeneous patient population. This assessment will be performed for each patient and is based on a series of defined criteria identified through an extensive literature review of the prognostic factors predictive of an unfavourable outcome after treatment with intensive chemotherapy combination with different schedules of cytarabine and anthracycline

Within the present invention the term “AML” is to be understood to encompass all forms of acute myeloid leukemia and related neoplasms according to the 2008 revision of the World Health Organization (WHO) classification of myeloid neoplasms and acute leukemia. Further all above mentioned subgroups in their relapsed or refractory state are encompassed. These are:
Acute myeloid leukemia with recurrent genetic abnormalities
   AML with t(8;21)(q22;q22); RUNX1-RUNX1T1
   AML with inv(16)(p13.1q22) or t(16;16)(p13.1;q22); CBFB-MYH11
   AML with t(9;11)(p22;q23); MLLT3-MLL
   AML with t(6;9)(p23;q34); DEK-NUP214
   AML with inv(3)(q21 q26.2) or t(3;3)(q21;q26.2); RPN1-EVI1
   AML (megakaryoblastic) with t(1;22)(p13;q13); RBM15-MKL1
   Provisional entity: AML with mutated NPM1
   Provisional entity: AML with mutated CEBPA
Acute myeloid leukemia with myelodysplasia-related changes
Therapy-related myeloid neoplasms
Acute myeloid leukemia, not otherwise specified
   AML with minimal differentiation
   AML without maturation
   AML with maturation
   Acute myelomonocytic leukemia
   Acute monoblastic/monocytic leukemia
   Acute erythroid leukemia
      Pure erythroid leukemia
      Erythroleukemia, erythroid/myeloid
   Acute megakaryoblastic leukemia
   Acute basophilic leukemia
   Acute panmyelosis with myelofibrosis
Myeloid sarcoma
Myeloid proliferations related to Down syndrome
   Transient abnormal myelopoiesis
   Myeloid leukemia associated with Down syndrome
Blastic plasmacytoid dendritic cell neoplasm

Preferably the term “AML” is to be understood to mean any of the AML subtypes mentioned above.

In accordance with the present invention Volasertib may be administered parenterally by infusion or injection (e.g. intramuscular, intraperitoneal, intravenous, transdermal or subcutaneous), and may be formulated, alone or together, in suitable dosage unit formulations containing conventional non-toxic pharmaceutically acceptable carriers, adjuvants and vehicles appropriate for each route of administration. Dosage forms and formulations of one or more actives suitable within the present invention are known in the art. For instance, such dosage forms and formulations include those disclosed for Volasertib in WO 2006/018221.

In accordance with the present invention cytarabine may be administered by parenteral routes of administration (e.g. intramuscular, intraperitoneal, intravenous, transdermal or subcutaneous injection/infusion, or by implant. It may be formulated, alone or together, in suitable dosage unit formulations containing conventional non-toxic pharmaceutically acceptable carriers, adjuvants and vehicles appropriate for each route of administration.

In accordance with the present invention fludarabine may be administered by parenteral routes of administration (e.g. intramuscular, intraperitoneal, intravenous, transdermal or subcutaneous injection/infusion, or by implant). It may be formulated, alone or together, in suitable dosage unit formulations containing conventional non-toxic pharmaceutically acceptable carriers, adjuvants and vehicles appropriate for each route of administration.

In accordance with the present invention GCSF may be administered by parenteral routes of administration (e.g. intramuscular, intraperitoneal, intravenous, transdermal or subcutaneous injection/infusion, or by implant). It may be formulated, alone or together, in suitable dosage unit formulations containing conventional non-toxic pharmaceutically acceptable carriers, adjuvants and vehicles appropriate for each route of administration.

In accordance with the present invention daunorubicin citrate liposome may be administered by parenteral routes of administration (e.g. intramuscular, intraperitoneal, intravenous, transdermal or subcutaneous injection/infusion, or by implant). It may be formulated, alone or together, in suitable dosage unit formulations containing conventional non-toxic pharmaceutically acceptable carriers, adjuvants and vehicles appropriate for each route of administration.

1. A method of treating acute myeloid leukemia (AML) comprising administration to a patient suffering from AML
a) an effective amount (50-500 μg/m² body surface area) of GCSF at day 0, 1, 2, 3, 4 and 5 during a 6 day treatment cycle (days 0 to 5);
b) an effective amount (10-100 mg/m² body surface area) of fludarabine at day 1, 2, 3, 4 and 5 during said 6 day treatment cycle;
c) an effective amount (500-4000 mg/m² body surface area) of cytarabine at day 1, 2, 3, 4 and 5 during said 6 day treatment cycle; and
d) an effective amount (10 to 350 mg/m² body surface area) of Volasertib, or a pharmaceutically acceptable salt or hydrate thereof, at least one day up to 5 days during said 6 day treatment cycle.

2. A method of treating acute myeloid leukemia (AML) comprising administration to a patient suffering from AML
a) an effective amount (50-500 μg/m² body surface area) of GCSF by i.v. infusion at day 0, 1, 2, 3, 4 and 5 during a 6 day treatment cycle (days 0 to 5),
b) an effective amount (10-100 mg/m² body surface area) of fludarabine at day 1, 2, 3, 4 and 5 during said 6 day treatment cycle,
c) an effective amount (500-4000 mg/m² body surface area) of cytarabine at day 1, 2, 3, 4 and 5 during said 6 day treatment cycle,
d) an effective amount (500-4000 mg/m² body surface area) of daunorubicin citrate liposome at day 1, 3 and 5 during said 6 day treatment cycle, and
e) an effective amount (10 to 350 mg/m² body surface area) of Volasertib, or a pharmaceutically acceptable salt or hydrate thereof, at least one day up to 5 days during said 6 day treatment cycle.

3. The method of claim 2 comprising administration to a patient suffering from AML
a) an effective amount (200 μg/m² body surface area) of GCSF by i.v. infusion at day 0, 1, 2, 3, 4 and 5 during a 6 day treatment cycle (days 0 to 5),
b) an effective amount (30 mg/m² body surface are) of fludarabine at day 1, 2, 3, 4 and 5 during said 6 day treatment cycle,
c) an effective amount (2000 mg/m² body surface area) of cytarabine at day 1, 2, 3, 4 and 5 during said 6 day treatment cycle,
d) an effective amount (2000 mg/m² body surface area) of daunorubicin citrate liposome at day 1, 3 and 5 during said 6 day treatment cycle, and
e) an effective amount (10, 50, 100, 150, 200, 250, 300, or 350 mg/m² body surface area) of Volasertib, or a pharmaceutically acceptable salt or hydrate thereof, at least one day up to 5 days during said 6 day treatment cycle.

4. A pharmaceutical composition comprising an effective amount of fludarabine, cytarabine and GCSF optionally together with an instruction for administration of the active ingredients to a patient suffering from AML, wherein the composition additionally comprises an effective amount of Volasertib or a pharmaceutically acceptable salt or hydrate thereof.

5. The pharmaceutical composition according to claim 4 further comprising an effective amount of daunorubicin citrate liposome.
